(19) United States
(12) Patent Application Publication
(10) Pub. No.: US 2023/0301827 A1
(43) Pub. Date: Sep. 28, 2023

(54) Spray Device

(71) Applicant: Medspray B.V., Enschede (NL)

(72) Inventors: Cornelis Johannes Maria Van Rijn, Amsterdam (NL); Franciscus Martinus Verhoeven, Enschede (NL); Wietze Nijdam, Enschede (NL)

(21) Appl. No.: 18/021,894

(22) PCT Filed: Aug. 17, 2021
(86) PCT No.: PCT/IB2021/057558

§ 371 Date: Feb. 17, 2023

(30) Foreign Application Priority Data
Aug. 17, 2020 (NL) 2026282

(51) Int. Cl. A61F 9/00; B05B 1/14

(52) U.S. Cl. CPC A61F 9/0008; B05B 1/14

(57) ABSTRACT

A spray device for delivering a liquid spray, having a spray length, through air to a target, in particular an eye, comprises a container for holding a liquid, means for pressurizing a quantity of said liquid to an elevated operating pressure, and comprises a nozzle member that comprises a nozzle plate with a plurality of nozzle orifices of substantially identical size, extending through said nozzle plate and communicating with said means to receive a quantity of pressurized liquid at said operating pressure. Said nozzle orifices discharge said liquid spray at an outlet surface of said nozzle member member over said spray length Their substantially identical size is between lower and upper limits (Dmin), (Dmax),
wherein said lower limit is approximately:

$$D min \approx 1 / 10 \sqrt{\frac{L λ}{\sqrt{Δ P / ρ}}} ,$$

wherein said upper limit is approximately:

$${D}_{max} \approx 1 / 10 \sqrt{\frac{2 L λ}{\sqrt{Δ P / ρ}}} .$$

λ≈18 η/ρ, η=1,8.10⁻⁵ kg/ms, representing the viscosity of air and ρ representing a density of said liquid.

The present invention relates to a spray device for delivering a spray of a liquid having a density (p) at a spray length (L) through air to a target, in particular to an eye of a user, comprising a container holding said liquid, pressurizing means configured for pressurizing a quantity of said liquid to an elevated operating pressure (P), and comprising a spray nozzle member with a plurality (N) of nozzle orifices extending from an inlet surface to an outlet surface of a nozzle plate, and wherein said nozzle member communicates with said pressurizing means, during operation, exposing said plurality of orifices at said inlet surface to said quantity of pressurized liquid at said operating pressure and releasing said spray at said outlet surface. The invention further relates to a method for delivering a spray of a liquid having a density (p) through air to a target at a distance (L), in particular to an eye of a user, comprising: providing a quantity of said liquid having said density (ρ); pressurizing said quantity of said liquid to an elevated operating pressure (P), and forcing said liquid at said elevated pressure (P) through a plurality (N) nozzle orifices.

Current available eye spray devices are being used to produce spray droplets with a broad droplet size distribution, typically between 10 and 100 micron with a large Geometric Standard Deviation (GSD»1.6). Spray characteristics are determined by a number of factors including the dimension and geometry of the outlet nozzle and the pressure with which the fluid is forced through the nozzle. It is known that sprays with uniformly sized small droplets are difficult to produce. Uniform sized droplets are however beneficiary if one aims at low impact sprays, that are homogeneously distributed over the target area hitting the target with a uniform velocity. A droplet of 20 micron has 8 times more mass than a droplet of 10 micron, and a droplet of 20 micron will therefore decelerate much less in air and hit the target with a larger velocity than a droplet of 10 micron. Current sprays having a rather broad size distribution, typically between 10 and 100 micron are thus also characterized by droplets with a broad velocity distribution.

The generation of a uniform low impact spray is becoming particularly desirable when it would be enabled by low pressure pumps, such as the manually-operable pump or trigger sprays being used for many over-the-counter (OTC) sprays. However current OTC spray devices, producing droplets with a typical size of 10-100 micron, are generated with so called pressure swirl nozzles or hollow cone nozzles. A stationary core inside the nozzle induces a rotary fluid motion which causes the swirling of fluid in a swirl chamber. A liquid sheet film is discharged from the perimeter of the outlet orifice producing a characteristic hollow cone spray pattern. Air or other surrounding gas is drawn inside the swirl chamber to form an air core within the swirling liquid.

Many geometries of fluid inlets are used to produce this hollow cone pattern depending on the nozzle capacity and materials of construction. These OTC nozzles still produce droplets with a rather broad size distribution sizes typically between 10-100 micron, and a mean drop size of 40-60 micron. Therefore, to generate a cone spray with uniformly sized droplets at a low working pressure below e.g. 10 bar has thus been proven quite difficult. Moreover, the minimum volume flow or discharge rate of sprays generated with swirl nozzles is high, typically larger than 200 ul/sec, and the corresponding impact of the spray liquid on the target area is always rather high.

An area where uniform low impact spray devices are especially demanded is in the delivery of eye medication. The application of fluids, as in the case of eye drops, in the eye has always posed a challenge. Particularly the use of high impact swirl nozzles will trigger blinking at the critical moment, causing the spray droplets to land on the eyelid, nose or other parts of the face instead of the intended target on the eyeball. The impact of a substantial volume of large droplets of fluid on the eyeball, tends to produce the blinking reaction. Additionally, a large volume of the medication flows out of the eye or is washed away by a common tearing or blinking reflex. As a result, the traditional method of administration turns out to be both inaccurate and wasteful. The swirl nozzle technology does not provide a satisfactory way of controlling the amount of medication that is dispensed, nor does it provide a way of ensuring that the medication that is dispensed actually lands on the eye and remains on the eye.

Accordingly, there is a need for a spray device for ophthalmic use, that is capable of delivering a more accurate dosage to a subject's eye without substantially triggering an eye reflex.

To that end a spray device and a method of the type as described in the opening paragraph, according to the present invention, are characterized in that said nozzle orifices have a substantially identical size (D_{nozzle}) that is between a lower limit (Dₘᵢₙ) and an upper limit (Dₘₐₓ), wherein said lower limit is approximately:

$${D}_{min} \approx 1 / 10 \sqrt{\frac{L λ}{\sqrt{P / ρ}}} ,$$

and wherein said upper limit is approximately:

$${D}_{max} = 1 / 10 \sqrt{\frac{2 L λ}{\sqrt{P / ρ}}} ,$$

wherein λ≈18 η/ρ, and η=1,8.10⁻⁵ kg/ms, representing the viscosity of air, p represents the density of said liquid, P represents said operating pressure and L represents the spray length of the device.

Said size of an orifice is being defined in the present application as representing the diameter of a circle having a same surface area as a cross sectional surface area of said orifice. The present invention particularly relates to a spray device for generating a so-called micro-jet spray. A micro-jet spray consists of a number of concurrently emitting jets, in which each jet will initially breakup into a mono-disperse primary droplet train according to a jet breakup mechanism. As a result, consecutive primary droplets have a same size and propagate from the nozzle orifice in a same direction, typically the diameter of the primary droplet is between 1,85 and 2 times the diameter of the nozzle orifice.

The liquid is released by said orifices as (N) concurrently emitting jets that breakup into individual droplets of substantially identical size and substantially identical initial velocity. By choosing the size of said orifices between said lower limit and said upper limit, it is established that substantially all droplets will hit the target at a substantially equal terminal velocity. Said terminal velocity will have an average value of between 10% and 50% of the initial velocity of the emitted jets. The amount of said orifices and, hence, the number (N) of said jets is preferably chosen between 10 and 100, and more particularly between 10 and 50 orifices.

It is an insight according to the invention that the triggering of the blink reflex can be prevented, by lowering the impact of the spray liquid on the target area, especially when the droplet size is substantially less than 50 micron and that the volume flow or discharge rate is substantially less than 200 ul/sec, in particular less than 100 ul/sec.

To that end, a preferred embodiment of the spray device is thereby characterized in that said size of said orifices is less than 10 micron, in that said pressurizing means pressurize a quantity of between 5 and 50 microliter of said liquid to an operating pressure of between 5 and 15 bar, and in that nozzle orifices discharge said quantity of pressurized liquid over a period of at least half of a second.

The invention is thereby based on the recognition that the triggering of the blink reflex can be prevented, by setting a maximum on the concurrent impact of the spray liquid on the target area. Surprisingly it has been found that not only the discharge rate of the liquid is an important factor, but that also the induced force of spray impact on the target, being responsible for the blink reflex. The force of spray impact on the target is here defined as the total quantity of deposited mass multiplied with the spray velocity hitting the target divided by the discharge period of the spray device.

Concerning the force of spray impact, it is an insight according to the invention that, depending on their size, the spray droplets will decelerate substantially from their initial velocity before they hit the target at their terminal velocity. Especially, it has been found that small droplets decelerate much faster in air than larger droplets. When a droplet diameter is reduced by half, it turns out that the deceleration of such droplet will be about four times stronger. This will greatly reduce the terminal velocity of the droplets on impact on the target. At the same time the decelerated droplets in a droplet train may also tend to coalesce with subsequent droplets that are in their slip stream. The inventors have recognized that the latter may lead to a growth on average five times the size of the droplets, which will also increase the spray length of the device. The operating pressure and orifice size of the spray device together determine the droplet size (mass) and initial velocity and, hence, their initial and terminal momentum. The upper limit Dmax ensures that the latter will not exceed a threshold that will trigger a blinking reflex of the eye.

On the other hand a maximum travelling distance and terminal velocity of the droplets need be sufficient to reach and contact the target area, i.e. the eyeball. To that end the initial momentum of the droplets should be sufficiently high, which is assured by the lower limit Dmin of the orifice size that inter alia determines the initial velocity as a result of the spray pressure (P) and initial droplet size produced by the orifice diameter.

It has been recognized that the time dependent traveling distance x(t) and velocity v(t) of the droplets scale exponentially in time. At a typical initial velocity of v(t=0)=vₒ m/s their time dependent droplet velocity v(t) and time dependent travel distance x(t) are given by:

$$v ( t ) \approx {v}_{o} {e}^{- \frac{λ t}{{D}_{drop}^{2}}} and x ( t ) x ( t = 0 ) \approx \frac{{v}_{o} {D}_{drop}^{2}}{λ} ( \begin{matrix}1 & {e}^{- \frac{λ t}{{D}_{drop}^{2}}}\end{matrix} )$$

by first solving v(t) in Stokes law:

$$\frac{ρ π {D}_{drop}^{3}}{6} \frac{\partial v ( t )}{\partial t} = 3 η {D}_{drop} v ( t ) ,$$

that describes the movement of a single droplet in a surrounding fluid like air wherein λ≈18 η/ρ, η the air viscosity, ρ the liquid density and D_{drop} the droplet diameter. For a single droplet with diameter Ddᵣₒₚ=10 micron, λ≈32.4×10⁻⁸ m²s⁻¹ and at an initial velocity vₒ=30 m/s this leads to a maximum travel distance

$${L}_{max} = \frac{{v}_{o} {D}_{drop}^{2}}{λ} = \frac{ρ {v}_{o} {D}_{drop}^{2}}{18 η}$$

of about 1 cm.

For micro-jet sprays the maximum travel distance Lmax will be co-determined due to the traveling of the droplets in a droplet train, due to droplet coalescence inside the travel train, and also to a contribution from entrained air flow around the droplet train. To compensate for this effect a practical approach is that a spray consisting of many interacting droplets in a train with initial diameter Dₜᵣₐᵢₙ is assumed to behave as a spray of non-interacting single droplets with an effective Stokes diameter D_{single}. In practice the ratio D_{single}/Dₜᵣₐᵢₙ≈5. In other words, the propagation of a droplet train of primary droplets with diameter Dₜᵣₐᵢₙ=10 micron is considered as the propagation of a single droplet with diameter 50 micron. The maximum travel distance Lmax of a droplet train of primary droplets with diameter Dₜᵣₐᵢₙ=10 micron at an initial velocity vₒ=30 m/s would be (D_{single}/Dₜᵣₐᵢₙ)²×1 cm=25 cm. An effective operating pressure P over the nozzle plate is typically about 10 bar. If all of the operating pressure is transferred to kinetic energy, Bernoulli equation applies, stating P=ρvₒ², and find vₒ=30 m/s. Hence, the initial velocity of the jet ejected from the nozzle is typically about 30 m/s.

The preferred droplet velocity v_{T} for low impact on the target should be below 10 m/s at a target spray length L of between 5 and 10 cm, that is typical for an eye spray. For a given maximum spray length Lₘₐₓ the initial velocity vₒ has dropped a factor 2 at half the distance Lₘₐₓ/2. So if the aim is to deliver a low impact spray by decelerating the initial velocity to below 10 m/s, i.e by at least by said factor 2, then the target should be placed at a distance L=Lₘₐₓ/2, corresponding to single drops with a size

$${D}_{single} = \sqrt{\frac{36 L η}{\sqrt{ρ P}}} .$$

However smaller drops are still being able to reach the spray length L, although it with much lower velocity. The smallest single drops that are still able to travel a distance L is given by

$${D}_{single} = \sqrt{\frac{18 L η}{\sqrt{ρ P}}} .$$

So all single drops in the size range

$$\sqrt{\frac{18 L η}{\sqrt{ρ P}}} < {D}_{single} < \sqrt{\frac{36 L η}{\sqrt{ρ P}}}$$

are able to reach the target placed at a spray length L with a velocity less than half of the initial velocity, and this formula thus defines the concept of spray length L and also implies an optimum required size range for the droplets. For droplets travelling in a train as in a micro-jet spray the said ratio D_{single}/Dₜᵣₐᵢₙ≈5 should be used. At an operating pressure of 10 bar and setting the target at a distance T=L(=Lₘₐₓ/2) to 10 cm and using the ratio D_{single}/Dₜᵣₐᵢₙ≈5, then substitution in above equation yields 7 μm<Dₜᵣₐᵢₙ<10 μm for an eye micro-jet spray (with η≈1.8×10⁻⁵ kg/ms, ρ=1000 kg/m³). Droplets smaller than 7 μm will never hit the target and droplets larger than 10 μm will not decelerate sufficiently to below 10 m/s, so that v_{T}<vₒ/2, whereas a typical preferred range for the droplet velocity at the target is 0.1vₒ<v_{T}<0.5 vₒ. For a micro-jet spray the droplet size is about two times the nozzle diameter D_{nozzle} thus Dₜᵣₐᵢₙ=2 D_{nozzle}. Thus for a low impact eye spray with a spray length of 10 cm we get then 3.5 μm<D_{nozzle}<5 μm as an operating window size of the nozzle diameter at an operating pressure of 10 bar.

Hence, more generally, at an operating pressure P over the nozzle plate, the present invention provides a spray device for delivering a liquid spray to a target at a given spray distance L of droplets emanating from one or more orifices having a substantially identical diameter D_{nozzle}, while securing a liquid a velocity at spray distance L less than typically around 10 m/s if the diameter D_{nozzle} is chosen in between the above range of Dmin to Dmax at an operating pressure of 10 bar.

In a particular embodiment the device according to the invention is characterized in that said nozzle orifices are of a substantially identical size with a diameter D_{nozzle} less than 20 micron, in that said pressurizing means generate an operating pressure P over the nozzle plate, in that said number N of orifices discharge said quantity V of pressurized liquid at a rate of between 10 and 100 microliter per second during at least T>500 microseconds, and that the said nozzle diameter D_{nozzle} is chosen between

$$\sqrt{\frac{18 L η}{\sqrt{ρ P}}} < 2 {AD}_{nozzle} < \sqrt{\frac{36 L η}{\sqrt{ρ P}}}$$

A=D_{single}/Dₜᵣₐᵢₙ has typically a value of 5, but in practice will also depend on the amount of entrained air, the number of droplets and the amount of divergence of the droplet train such that in practice it may be in a range of between 3-7.

With preference the pressurizing means comprises a manually operable pump having a piston to pressurize said liquid, wherein said quantity of liquid V is pressurized to an operating pressure P over the nozzle plate in one stroke of said piston. This method is seen as most cost, user and environment friendly for the OTC market.

The spray device according to the invention is advantageously used to create a low impact spray for ophthalmic and other beauty and home care applications. It has been found that, not only the discharge velocity, but also the total Force of spray impact on the target is important. The Force of spray impact (FSI) on the target is here defined as the total quantity of deposited mass m≡ρ V multiplied with the spray velocity hitting the target with velocity v_{L} divided by the discharge period T of the spray device, so FSI=ρ V·v_{L}/T.

To create a more uniform spray at a reduced Force of spray impact, it is an insight according to the invention to decelerate substantially the initial velocity of the ejected droplets by using droplets that all have a substantially identical size so that all droplets have the same terminal velocity when hitting the target.

Eye spray trials with test persons have revealed that inducing a Force of spray impact on the eye less than 2×10⁻⁴ kgm/s² will prevent a blink reflex. Therefore, a preferred embodiment of the spray device of the invention is characterized by means that maintain a Force of spray impact (FSI) below about 2×10⁻⁴ kgm/s².

For comparison conventional swirl nozzles typically operate at a discharge rate well over 40 microliter per 0.2 second, thus at a discharge rate of more than 200 microliter per second. The Force of spray impact on the target of swirl nozzles exceeds therefor 2×10⁻³ kg·m/s² at impact velocities of typical >10 m/s herewith easily triggering the blink reflex.

There is a delicate balance between several relevant parameters. In particular changing the mean droplet size and droplet size distribution of the spray has a profound influence on the impact force of the spray droplets at the target. Sprays with a narrow droplet size distribution are more suited for the creation of uniform low impact sprays.

Normally the droplet size distribution may be characterized in terms of volume as DVX, with X % being the total volume of liquid sprayed drops with a specific diameter expressed in micrometres (μm) smaller than DVX, and 100-X % of droplets with a larger diameter than DVX. A DV10 of 10 micron means that 10% of the spray volume has droplets with a diameter smaller than 10 micron. DV50 is also defined as the Volume Mean Diameter. The droplet size distribution is characterized by the Relative Span (RS) as RS≡(DV90−DV10)/DV50. Satisfactory uniform low impact eye sprays are delivered with a particular embodiment of the spray device according to the invention, characterized in that RS<1, in particular RS<0.5. Measured droplet size distributions by a further particular embodiment of a spray device according to the invention are further characterized by a Geometric Standard Deviation GSD<1.6, in particular GSD<1.4. These eye sprays can be considered as nearly monodisperse.

The invention, moreover, relates to a method for delivering a liquid spray to a target at a spray distance L, in particular to an eye, comprising: pressurizing a quantity of said liquid to an elevated operating pressure, forcing said liquid at said elevated pressure at an initial velocity through a plurality (N) of nozzle orifices that are provided in a nozzle plate of a spray nozzle, herewith generating a micro-jet spray, consisting of (N) concurrently emitting jets that breakup in droplets, characterized in that a substantial part of the droplets hit the target at a substantial equal velocity, which an (average) value between 10% and 50% of the initial velocity of the emitted jets, and that N is typical between 10 and 100.

Further investigations and experiments revealed that for eye sprays, according to the invention, the nozzle orifices preferably have a nozzle orifice opening of between 3-6 micron in size, in particular between 3.5 and 5 micron, creating primary ejected droplets between 6-12 micron and downstream droplets with a size of 20-40 micron.

Preferably the spray emanating from the spray device is homogenously distributed over a specific target area, such as the eyeball or a specific skin area, and that the user should be able to direct the spray to the target area. A further preferred embodiment of the spray device according to the invention, to that end, is capable of producing directed diverging rays characterized by a diverging ray angle that is typically between 5 and 25 degrees, said ray angle preferably being tunable.

Increasing the number of orifices will balance the required discharge rate, the given initial droplet velocity and the preferred optimum droplet/orifice size to provide a uniform spray of sufficiently low impact. For eye spray purposes, typically 10-50 diverging rays appear to provide this balance.

Of importance to the invention is that the nozzle device is held at a correct distance (L) from the target. To that end, a special embodiment of the spray device according to the invention is characterized in that that a first object is provided at a first distance from said outlet surface, in that a second object is provided at a second distance from said outlet surface, said second distance being larger than said first distance, and in that said second image matches said first image in a common focal plane of the eye of a user when said outlet surface is at said spray length (L) spaced apart from said eye. This embodiment is based on the recognition that the user will observe said matching objects via the pupil of the eye only if said spray device is held with said outlet surface at said distance L from the eye. Only in that case the second object exactly matches with the first object and an optimal spray distance is reached. At that position, the spray device is preferably operated for spraying. The objects may be physical objects or graphical representations. The objects may be said to match when a circumference of one of the objects coincides with a similar, corresponding, complementary or otherwise uniquely associated circumference of the other object. Said first distance and said second distance may be zero or negative as long as they are different from one another to define a unique common focal plane at said distance L at which both objects, being either physical or graphical, exactly match.

Without additional measures, uniform droplets made by Rayleigh jets will collide and form larger droplets. All droplets move in the original jet direction. Droplets are slowed down by friction and trailing droplets moving in the slipstream of each other have less friction and thus move faster until they collide with the leading droplet in front of them. This effect is self-enhancing, because collided droplets are larger and thus have more friction with the surrounding air, are slowed down even more and thus even more droplets will collide. This may adversely affect the force of impact they will have at the target.

In order to avoid such coalescence, a further special embodiment of the spray device according to the invention is characterized in that an electro-acoustical transducer device is provided in the vicinity of said outlet surface that is configured to generate, when energized, a longitudinal soundwave propagating in a direction crossing a propagation direction of said liquid spray and to expose said liquid spray to the same, particularly substantially perpendicularly. In a corresponding method of the invention the liquid spray that is being released is exposed to said longitudinal soundwave that will move the droplets out of their trajectory more or less randomly in order to avoid a common propagation path. Thus it is counteracted that trailing droplets will hit leading droplets in a single spray jet.

### BRIEF DESCRIPTION OF THE DRAWINGS

FIG. 1 illustrates a spray device for delivering a liquid spray of droplets.

FIG. 2 illustrates a spray nozzle unit having a nozzle plate with a number of nozzle orifices.

FIG. 3 shows a movie frame of a high speed camera of a spray.

FIG. 4 shows a velocity profile of a spray.

FIG. 5 shows a specific embodiment of the device and method according to the invention.

FIG. 6 shows a further specific embodiment of the device and method according to the invention.

FIG. 7 shows a further specific embodiment of the device and method according to the invention.

FIG. 8 shows a further specific embodiment of the device and method according to the invention.

FIG. 9 shows a further specific embodiment of the device and method according to the invention.

FIG. 10 shows a further specific embodiment of the device and method according to the invention.

FIG. 11 shows a further specific embodiment of the device and method according to the invention.

### DESCRIPTION OF THE PREFERRED EMBODIMENTS

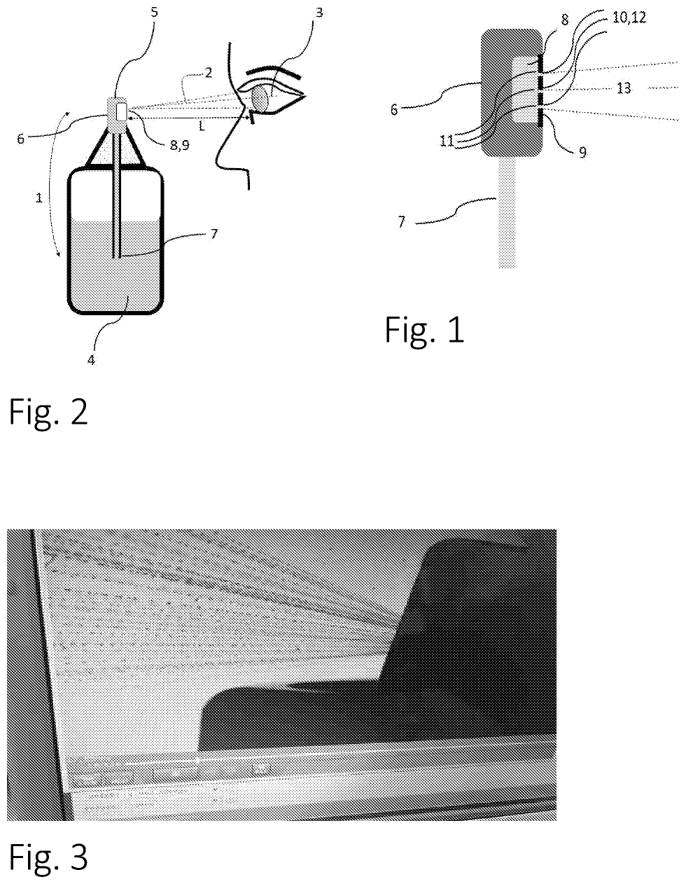

FIG. 1 illustrates a spray device (1) for delivering a liquid spray of droplets (2) with an initial velocity vₒ larger than 10 m/s to an eye (3) at a given distance L, hitting the eye (3) at a strongly reduced velocity v_{L} The spray device (1) comprises a container (4) for holding the spray liquid, pumping means (5) for pressurizing a quantity V=15 microliter, a spray nozzle unit (6) having an inlet (7) and having an outlet (8), wherein the spray nozzle unit (6) produces a spray (2), during operation for a period T=1 sec.

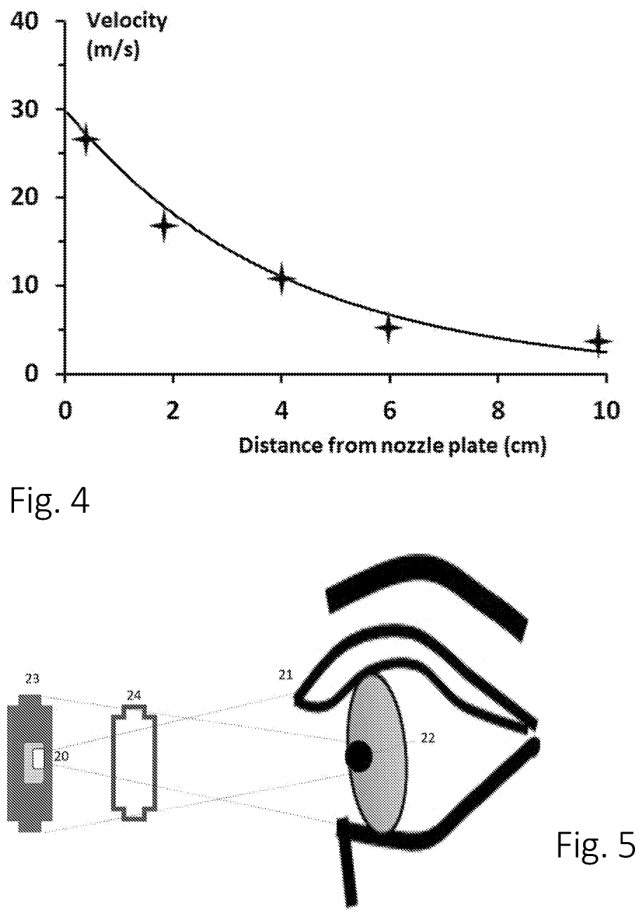

FIG. 2 illustrates a spray nozzle unit (6) having an inlet (7) and outlet (8), and comprising a nozzle plate (9) having a number (10) of nozzle orifices, each having an entrance (11) in open communication with said inlet (7) and having an exit (12) in open communication with said outlet (8). The nozzle orifices (10) have a substantially identical size, here of 4.5 micron producing a droplet train (13) with a droplet diameter Dₜᵣₐᵢₙ≈9 micron. The pressurizing means at an operating pressure P≈10 bar over the nozzle plate (9) generate jets with an initial velocity of vₒ=30 m/s originating from 40 nozzle orifices (10). In T=0.5 sec a total quantity V=10 microliter at a rate of 20 microliter per second is then discharged. With an ultra-high speed Shimadzu camera the velocity profile of the 40 diverging jets as depicted in FIG. 3 has been obtained and is plotted in FIG. 4. The from this experiment derived Stokes diameter D_{single} is here ca. 45 micron, thus 5 times the initial droplet train diameter. At a distance of 5 cm from the nozzle plate the velocity has dropped about with a factor e=2.7 from 28 m/s to ca 10 m/s. Thus for this configuration according to the invention the target should be placed between L=5 and 10 cm from the nozzle plate (9) using 50 orifices (10) with a nozzle diameter of 4.5 micron. In order to prevent the blinking reflex when the spray is directed towards the open eye the discharge rate should be less than 50 ul/sec and the Force of Spray Impact FSI=ρ V·v_{L}/T should be equal or less than 2×10⁻⁴ kgm/s². In this case the discharge rate is 20 ul/sec and at a distance of 5 cm the Force of Spray Impact FSI=2×10⁻⁴ kgm/s². Below some results of the eye spray devices with healthy volunteers are put in a table.

| | | | | | | Force of | | |
|---|---|---|---|---|---|---|---|---|
| | | | Distance | Discharge | | Spray | Triggering | No Triggering |
| Test | Number | | to Eye | Rate in | Discharge | Impact | of Blink | of Blink |
| Number | Volunteer | Device type | in cm | uliter/sec | Time sec | kgm/s² | Reflex Count | Reflex Count |
| 1 | 8 | Swirl | 5 | 200 | 0.2 | 20 × 10⁻⁴ | 8 | 0 |
| 2 | 8 | Dₜᵣₐᵢₙ = 9 micron | 5 | 20 | 0.5 | 2 × 10⁻⁴ | 0 | 8 |
| 3 | 8 | Dₜᵣₐᵢₙ = 12 micron | 5 | 20 | 1.0 | 2 × 10⁻⁴ | 0 | 8 |
| 4 | 12 | Swirl | 7.5 | 200 | 0.2 | 15 × 10⁻⁴ | 12 | 0 |
| 5 | 15 | Dₜᵣₐᵢₙ = 9 micron | 7.5 | 20 | 0.5 | 1.3 × 10⁻⁴ | 0 | 15 |
| 6 | 13 | Dₜᵣₐᵢₙ = 12 micron | 7.5 | 20 | 1.0 | 1.3 × 10⁻⁴ | 1 | 12 |
| 7 | 10 | Swirl | 10 | 200 | 0.2 | 10 × 10⁻⁴ | 9 | 1 |
| 8 | 10 | Dₜᵣₐᵢₙ = 9 micron | 10 | 100 | 0.5 | 4 × 10⁻⁴ | 7 | 3 |
| 9 | 9 | Dₜᵣₐᵢₙ = 12 micron | 10 | 100 | 1.0 | 4 × 10⁻⁴ | 8 | 1 |

Three different eye spray devices have been used, two according to the invention and one conventional commercial swirl nozzle type device with a typical volume of 40 ul per stroke and a discharge time of 0.2 sec. According to the invention we have used two nozzle diameters, 4.5 and 6 micron respectively and two different number of nozzle orifices 40 and 200 respectively. The table shows that when both the Discharge Rate and Force of spray impact are relatively high that the blink reflex of the volunteers are easily triggered. To prevent the blink reflex it can be derived from the table that the Force of spray impact should be lower than 2×10⁻⁴ kgm/s² and also that the Discharge Rate is preferably substantially lower than 200 microliter per second.

A special embodiment of a spray device and method according to the invention is illustrated in FIG. 5. A spray nozzle member (20) ejects a micro-jet spray under a diverging ray angle to the eye or eye ball (21) of the user. A first object in the form of a graphical image (23) is printed or connected around the center of the spray nozzle member (20) at a first distance to the outlet surface at or close to zero where the liquid spray emanates. In this example this image has the shape of a solid cross (23).

A second object (24) is provided at a second distance from the outlet surface. This second object comprises an open window frame (24) that is connected or fixed to the spray device at a distance of typical between 2 mm and 2 cm in front of the outlet surface, hence, in front of the first object (23) and co-axial sharing a common center line. An inner circumference of this window (24) is exactly conformal to an outer circumference of the image (23) although slightly larger.

A microjet spray released by the spray device (20) will be able to pass through the open window (24) and will typically travel between 5 and 10 cm to reach the eye ball (21). Before starting spraying, the user looks via the pupil (22) of his eye (21) through the open window (24) to the first object (23). Once the user observes that the outer circumference of said image (23) exactly matches the inner circumference of the open window (24) an optimal spray distance L is reached, and the user may start operating the spray device for spraying. Both objects are now in a single focal plane of the eye.

The outer circumference of the image (23) is slightly larger than the inner circumference of the open window (24). The difference in circumference sizes together with the distance between them will determine and set the optimal spray distance L. Many different variations with respect to the first and second object are possible. For instance other shapes than a cross are possible for the circumference of said image (23) and said open frame (24), such as a circle, a star, a rectangle and the like. Variations the first and second object may also comprise a different color, or a fluorescent stain. Also the use of a LED light may be helpful for determining the optimal spray distance. The LED light image may for instance be given a shape of the first image (23).

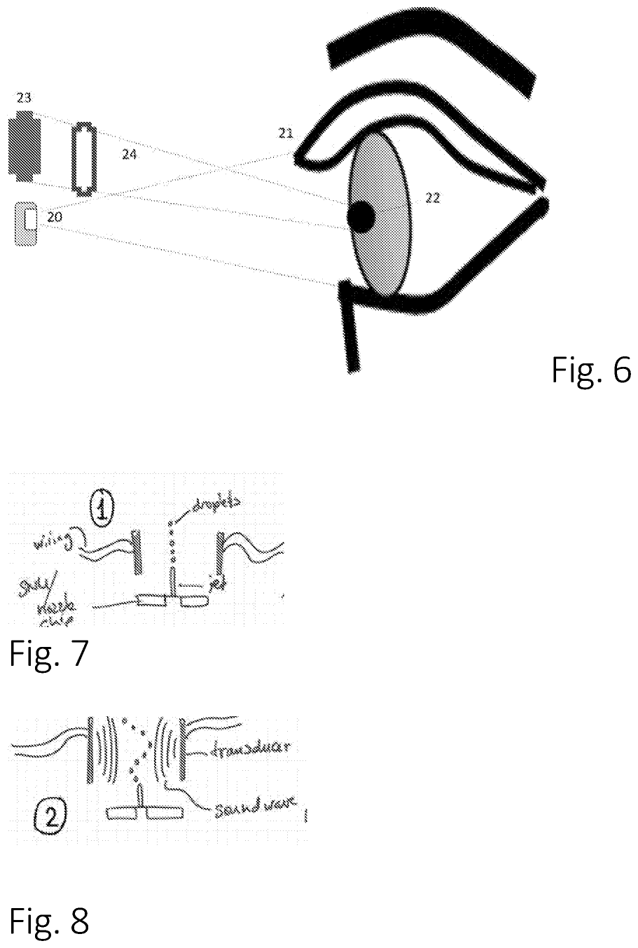

Likewise said image (23) and open frame (24) can also be placed at another position relative to the spray device, for instance away from the center of the spray nozzle member (20). As shown in the embodiment of FIG. 6 that further corresponds to the embodiment of FIG. 5.

A further special embodiment of the device and method according to the invention are depicted in FIG. 7. FIG. 7 shows a typical nozzle chip according to the invention spraying a liquid, thereby forming a liquid jets that breaks up in droplets by means of Rayleigh jet principle.

Without additional measures, the droplets will continue to move in their original propagation direction of the jet flow. In this embodiment, however, small transducers are placed at opposite sides of the jet/droplets in the vicinity of the outlet surface where they were released, i.e. near the jet break up area. When energized and as soon as longitudinal sound waves are generated, as shown in FIG. 8, the droplets will move with the moving air as long as they are in the sound wave.

The two parallel placed transducers may be coupled, such that their output is doubled and more directional. If the sound waves are in phase, standing waves will exist with regions that trap the droplets or with regions in which droplets will be expelled (anti-nodes/nodes). By applying different and/or varying frequencies these regions will move perpendicular to the jet propagation path and thus move the droplets out of their common trajectory.

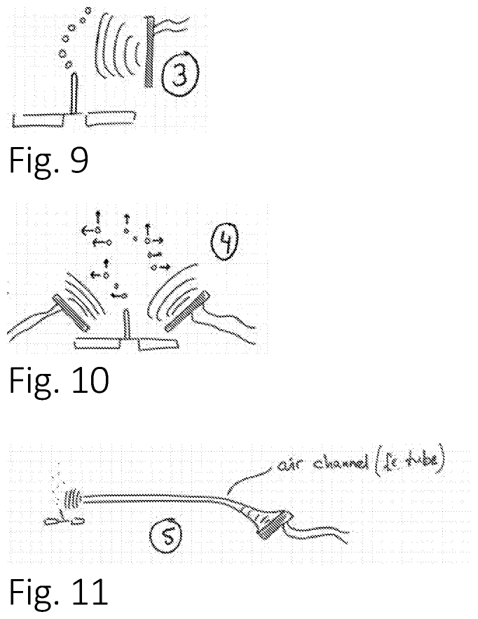

FIG. 9 shows an embodiment in which, instead of a pair of transducers, a single transducer is sufficient to move the droplets in air. Only small movements may be required in the order of 1-5 times the droplet size, in order to space the droplets apart from each other in a droplet train (slip stream) to avoid collision.

FIG. 10 shows a further embodiment by placing two transducers at an inclined angle with each other and relative to the jet. The inclined angle may be between 0 and 90 degrees. The generated sound wave will have a both a traverse and longitudinal component crossing the droplet train. The latter may propel the droplets (y-direction), while the former will displace them (x-direction) depending on the resulting wave front from the two colliding sound waves. This may moreover aid in widening the spray flume for better evaporation or for more aesthetic appearance.

Placing the transducers within the spray nozzle device or even within the spray nozzle member, i.e. directly in the vicinity of the outlet surface may prove difficult in case of insufficient space. Although preferably relatively small transducers are used, larger transducers may also be positioned further away from the jets. FIG. 11 shows such an embodiment. In this case an air channel is used to guide the longitudinal sound wave towards the jet break-up area of the Raleigh jets near the outlet surface.

Large droplets (large 30 μm droplets, low flow rate) typically break up with frequencies of 10 kHz and higher. On the other end of the spectrum, small droplets, for instance used for inhalation therapy, tend to break-up with frequencies of several MHz (up to 10 MHz). It may be beneficial, although not required, to have transducers operating at a transducer frequency similar to the break-up frequency of the spray droplets concerned.

Although the invention was described with reference to only a limited number of embodiments it will be appreciated that the invention is by no means limited to the examples that were given. On the contrary many more embodiments and variations are feasible to a skilled person within the scope of the present invention without requiring him or her to exercise any inventive labor or skill. In general the present invention provides for a unique aye spray device and method of spraying a liquid to the eye of a user that avoids the conventional blinking effect and associated nuisance.

1. A spray device for delivering a spray of a liquid having a density (φ at a spray length (L) through air to a target, in particular to an eye of a user, comprising a container holding said liquid, pressurizing means configured for pressurizing a quantity of said liquid to an elevated operating pressure (ΔP), and comprising a spray nozzle member with a plurality (N) of nozzle orifices extending from an inlet surface to an outlet surface of a nozzle plate, and wherein said nozzle member communicates with said pressurizing means, during operation, exposing said plurality of orifices at said inlet surface to said quantity of pressurized liquid at said operating pressure and releasing said spray at said outlet surface, wherein said nozzle orifices of said plurality of nozzle orifices have a substantially identical size (D_{nozzle}) that is between a lower limit (Dₘᵢₙ) and an upper limit (Dₘₐₓ),
said lower limit being approximately:
$$D min \approx 1 / 10 \sqrt{\frac{L λ}{\sqrt{Δ P / ρ}}} ,$$
said upper limit being approximately:
$${D}_{max} \approx 1 / 10 \sqrt{\frac{2 L λ}{\sqrt{Δ P / ρ}}} ,$$
in which λ≈18 η/ρ, η=1,8.10⁻⁵ kg/ms, representing the viscosity of air, and p represents said density of said liquid.

2. A spray device according to claim 1, wherein said nozzle orifices have a substantially identical size (D_{nozzle}) of less than 10 micron, wherein said pressurizing means are configured to pressurize a quantity of between 5 and 50 microliter of said liquid to an operating pressure of between 5 and 15 bar, and wherein said plurality of nozzle orifices discharge said quantity of pressurized liquid over a period of at least 500 microseconds.

3. Spray device according to claim 1, wherein the pressurizing means comprises a manually operable pump having at least one piston to pressurize said quantity of liquid, wherein said pump is configured to pressurize said quantity of liquid ΔV to said operating pressure ΔP in one stroke of said at least one piston.

4. Spray device according to claim 1, wherein said plurality (N) of spray nozzle orifices are configured to discharge a corresponding plurality of spray micro-jets that diverge mutually from said outlet surface, said plurality of nozzle orifices comprising particularly between 10 and 100 orifices, and more particularly between 10 and 50 orifices.

5. Spray device according to claim 4, wherein said plurality of spray micro-jets form a cone with a spray cone angle between 5° and 25°.

6. Spray device according to claim 1, wherein said spray nozzle member is configured to provide a force of spray impact FSI=ρ·ΔV·v_{L}/ΔT that is less than 1×10⁻³ kgm/s², in particular less than 2×10⁻⁴ kg·m/s², wherein v_{L} represents the impact velocity at spray length L.

7. Spray device according to claim 1, wherein said spray nozzle member is configured to have a discharge rate less than 50 ul/sec, while the spray length (L) is between 5 and 10 cm.

8. Spray device according to claim 1, wherein said orifices have a diameter of between 3 and 6 micron, in particular of between 3.5 and 5 micron, wherein, adjacent said orifices, a thickness of said nozzle plate is smaller than three times a diameter of said orifices, preferably smaller than said diameter.

9. Spray device according to claim 1, wherein a first object is provided at a first distance from said outlet surface, wherein a second object is provided at a second distance from said outlet surface, said second distance being larger than said first distance, and wherein said second image matches said first image in a common focal plane of the eye of a user when said outlet surface is at said spray length (L) spaced apart from said eye.

10. Spray device according to claim 1, wherein an electro-acoustical transducer device is provided in the vicinity of said outlet surface that is configured to generate, when energized, a longitudinal soundwave propagating in a direction crossing a propagation direction of said liquid spray and to expose said liquid spray to the same, particularly substantially perpendicularly.

11. A method for delivering a spray of a liquid having a density (ρ) through air to a target at a distance (L), in particular to an eye of a user, comprising:
providing a quantity of said liquid having said density (ρ);
pressurizing said quantity of said liquid to an elevated operating pressure (ΔP), and
forcing said liquid at said elevated pressure (ΔP) through a plurality (N) nozzle orifices,
characterized in that nozzle orifices of said plurality (N) of nozzle orifices have a substantially identical size (D_{nozzle}) that is between a lower limit (Dₘᵢₙ) and an upper limit (Dₘₐₓ),
said lower limit being approximately:
$$D min \approx 1 / 10 \sqrt{\frac{L λ}{\sqrt{Δ P / ρ}}} ,$$
said upper limit being approximately:
$${D}_{max} \approx 1 / 10 \sqrt{\frac{2 L λ}{\sqrt{Δ P / ρ}}} ,$$
in which λ≈18 η/ρ, η=1,8.10⁻⁵ kg/ms, representing the viscosity of air and ρ represents said density of said liquid, said nozzle orifices being provided in a nozzle plate of a spray nozzle member.

12. A method for delivering a liquid spray according to claim 11, wherein said quantity of said liquid is between 5 and 50 microliter and is released substantially continuously over a period of at least approximately 500 microseconds.

13. The method of claim 11, wherein said liquid is pressurized to an operating pressure of between 5 and 15 bar.

14. The method of claim 11, wherein said quantity of liquid is pressurized by means of a manually operable pump having at least one piston to pressurize said quantity of liquid, and wherein said quantity of liquid ΔV is pressurized to said operating pressure ΔP in one stroke of said at least one piston.

15. The method of claim 11, wherein said spray nozzle member is operated at a Force of Spray Impact FSI=ρ·ΔV·v_{L}/ΔT that is less than 1×10⁻³ kg·m/s², in particular less than 2×10⁻⁴ kg·m/s², wherein v_{L} represents the impact velocity at spray length L.

16. The method of claim 11, wherein said spray nozzle is operated at a discharge rate of less than 50 ul/sec.

17. The method of claim 11, wherein spray nozzle is held at a distance L of between 5 and 10 cm from said target, particularly from an eye of the user.

18. The method of claim 11, wherein the spray is characterized by a droplet size distribution with a Relative Span RS<1, in particular RS<0.5.

19. The method of claim 11, wherein the spray is characterized by a droplet size distribution with a Geometric Standard Deviation GSD<1.6, in particular a GSD<1.4.

20. The method of claim 11, wherein a first object is provided at a first distance from said outlet surface, wherein a second object is provided at a second distance from said outlet surface, said second distance being larger than said first distance, and wherein said outlet surface is held at a spray length (L) spaced apart from the eye of the user when said second image matches said first image in a common focal plane of the eye of a user.

21. The method of claim 11, wherein said liquid spray is exposed to a longitudinal soundwave that propagates in a direction crossing a propagation direction of said liquid spray, particularly substantially perpendicularly to said propagation direction of said liquid spray.
